(19) United States
(12) Patent Application Publication
(10) Pub. No.: US 2024/0016982 A1
(43) Pub. Date: Jan. 18, 2024

(54) APPARATUS FOR PREPARING A TISSUE GRAFT

(71) Applicant: CAREY TASCA PTY LTD, Caulfield South (AU)

(72) Inventors: Marcus Patrick CAREY, Carlton North (AU); Grace Joan CAREY, Fitzroy (AU)

(21) Appl. No.: 18/250,739

(22) PCT Filed: Aug. 25, 2021
(86) PCT No.: PCT/AU2021/050973

§ 371 Date: Apr. 26, 2023

(30) Foreign Application Priority Data
Oct. 26, 2020 (AU) 2020903865

(51) Int. Cl. A61L 27/36

(52) U.S. Cl. CPC A61L 27/3691; A61L 27/3604; A61L 27/367

(57) ABSTRACT

An apparatus for preparing a tissue graft comprises a cutting surface having a plurality of cutting edges arranged in an incision pattern, wherein the cutting surface is configured to prepare the tissue graft by incising at least part of the tissue graft according to the incision pattern, said incision pattern being configured to achieve expansion of at least part of the prepared graft.

### TECHNICAL FIELD

The present invention relates to apparatus for preparing a tissue graft for use in a medical procedure and a kit for preparing a tissue graft. It relates particularly, but not exclusively, to preparing a harvested fascia tissue, such as fascia lata or rectus sheath fascia by forming incisions in a pattern which allows the tissue or parts thereof to expand, for use in the surgical treatment or repair of medical defects or injury.

### BACKGROUND OF INVENTION

Procedures involving tissue grafts utilise tissue harvested from a donor to replace missing or damaged tissue in a patient, or repair a defect or injury. Tissue grafts may be autograft (moved from one tissue site to another on the same person), allograft (tissue is donated from one person for use in another person) or xenograft (tissue is donated from one species for use in another).

Skin grafting is commonly used if a patient has lost skin due to burns, injury or illness and most often involves use of an autograft. The required area of tissue is taken from another area of the patient where the skin is healthy. This often involves harvesting skin from the front outer thigh, abdomen, buttocks or back. In some cases, a split-thickness graft may be meshed so that less skin is required to be harvested from the donor site. Meshing the skin may involve the doctor punching multiple holes in the graft to stretch out the piece of graft skin. This also allows fluid to drain from under the skin graft. Skin graft meshing devices exist, such as the Meshgraft instrument from Zimmer Biomet which consists of a ratchet handle, continuous feed roller and a cutting roller. The harvested skin sheet is placed on a platform that is passed through the roller, and the full skin graft is meshed for use at the treatment site. These systems are effective at preparing harvested skin for use in the treatment of large areas of burns, for example. However, they are not effective at meshing other tissue types that do not possess comparable thickness or composition.

Surgical mesh made of inorganic and biological materials is used in a range of surgical procedures. The most common indication for using mesh is hernia surgery. However, mesh is frequently used in pelvic floor and reconstructive plastic surgery among other applications.

Pelvic organ prolapse (POP) is a condition in which the bladder, uterus and/or bowel protrude into or outside the vagina. POP is typically due to loss of the natural supports of the pelvic organs and the vaginal vault in women who had undergone a prior hysterectomy. POP and vaginal vault prolapse occurs after failure of direct and indirect supporting mechanisms and is frequently accompanied by weakness of the muscular pelvic floor and suspensory fibres of the parametrium and upper paracolpium. Stress urinary incontinence (SUI) is the involuntary loss of urine with physical activities such as coughing, sneezing, exercising, and lifting. Stress incontinence is usually due to poor support of the urethra but occasionally may be the result of dysfunction of the urethral sphincter muscle (often as the result of structural or nerve damage from pregnancy and childbirth or from previous anti-incontinence or prolapse surgery). POP and SUI are both highly prevalent conditions that often coexist. For women, the estimated risk of surgery for either condition by age 80 years is 20%.

Anterior and/or posterior colporrhaphy (native tissue repair) are the most commonly performed operations for POP. Sacral colpopexy is widely considered to be the gold standard operation for POP particularly in cases of recurrent vaginal prolapse and prolapse of the vaginal vault following hysterectomy. Vaginal vault prolapse occurs in approximately 10% of women following hysterectomy and occurs in equal numbers following abdominal and vaginal hysterectomy. The sacral colpopexy procedure can be performed through a laparotomy incision, laparoscopically or robotically.

Dissatisfaction with traditional colporrhaphy for POP resulted in increased usage of mesh to augment vaginal repair procedures in order to obtain higher success rates. However, studies have reported significant problems (e.g. pain, dyspareunia and mesh exposure) with the use of synthetic mesh during vaginal prolapse surgery. As at the priority date of this application, both the FDA (Food and Drug Administration, USA) and TGA (Therapeutic Goods Administration, Australia) have withdrawn approval for trans-vaginal synthetic mesh for POP and some synthetic mesh slings for SUI. Furthermore, the adverse publicity around the use of synthetic pelvic mesh to treat both prolapse and incontinence has resulted in a dramatic reduction of women undergoing surgery to treat POP and SUI. Increasingly, women who decide to undergo surgery for POP and SUI are requesting surgery that avoids the use of synthetic mesh.

Fascia lata is a suitable alternative graft to synthetic mesh when treating POP and SUI. Donor fascia lata from cadavers has been used to reinforce and give strength in reconstructive surgery, mostly for facial paralysis, and commonly for surgeries of the eye and ear. Autologous rectus sheath fascia has been used for many years to provide effective and durable treatment of female SUI. Autologous fascia lata has been used for many years in ophthalmology and plastic surgery. Typically, with ophthalmology and plastic surgery relatively thin strips of fascia lata are used. However, with surgery for POP much wider strips of autologous fascia lata are required resulting in increased morbidity at the donor site.

It would be desirable to overcome or at least ameliorate one or more of the problems with the prior art.

The discussion of the background to the invention included herein including reference to documents, acts, materials, devices, articles and the like is included to explain the context of the present invention. This is not to be taken as an admission or a suggestion that any of the material referred to was published, known or part of the common general knowledge in Australia or in any other country as at the priority date of any of the claims.

### SUMMARY OF INVENTION

Embodiments of the present disclosure provide apparatus and kits for preparation of a tissue graft, such as a fascia tissue graft by placement of a pattern of cuts that enable at least part of the graft to be expanded.

It is known to utilise fascia lata (FL) in the surgical treatment of POP e.g. by sacral colpopexy or transvaginal repair. However, it has not previously been contemplated to expand the FL tissue. It is possible this is owing to the fact that there was no perceived clinical need. More likely however, is absence of a suitable tool for achieving expansion of the delicate FL tissue without damaging or compromising its integrity. Existing tools and apparatus of the kind used to expand or “mesh” skin grafts are too large to prepare the significantly smaller FL grafts making it impossible to pass the small graft through the device. Moreover, compared to skin graft tissue, FL is thinner and more delicate with the consequence that existing skin meshers are likely to damage or weaken the FL graft.

Additionally, skin meshers produce large interstices to increase the total area of the graft by a ratio of up to 9:1. Expansion of this extent is possible by formation of long slits placed in relatively close columns. The skin thickness contributes to the strength of the meshed skin lattice. Since these large grafts are not used in load bearing applications, the expansion ratio is more important than the tensile strength of the graft. However in procedures that treat e.g. POP by sacral colpopexy or transvaginal repair, the load bearing performance or tensile strength of the graft is crucial. Meshing a FL graft using existing skin meshers, would likely diminish that performance producing unfavourable results. Furthermore, existing skin meshers only allow for meshing the entire graft—they do not permit conservation of part of the graft in an unmeshed form.

The present disclosure provides novel approaches to preparation of tissue grafts, particularly those that use fascia tissue such as fascia lata and rectus sheath fascia.

Viewed from one aspect, the present disclosure provides apparatus for preparing a tissue graft, the apparatus comprising a cutting surface having a plurality of cutting edges arranged in an incision pattern, wherein the cutting surface is configured to prepare the tissue graft by incising at least part of the tissue graft according to the incision pattern, said incision pattern being configured to achieve expansion of at least part of the prepared graft.

Preferably, the apparatus further comprises a bearing surface configured to be arranged in opposition to the cutting surface, wherein in use, the tissue graft is arranged between the cutting surface and the bearing surface and the cutting surface incises the tissue graft when pressure is applied to one or both of the bearing surface and the cutting surface.

The cutting surface may be a substantially flat surface or substantially convex surface. In embodiments providing a cutting surface and a bearing surface, one of the cutting surface and the bearing surface may be a substantially convex surface and optionally, the other of the cutting surface and the bearing surface is a substantially flat surface. In embodiments, where the convex surface of the cutting surface or the bearing surface is convex, the convex surface may be a roller and the roller may have a handle portion.

In some embodiments, the cutting surface is provided on a die pad with a handle portion adapted to be gripped by a user to apply pressure to incise the tissue graft. Two or more of the cutting surface, the die pad and the handle portion may formed from a unitary piece of material for ease of manufacture and/or recycling. Alternatively, they may be manufactured from different component parts which may be formed from different materials and assembled together to form a tool.

In some embodiments, the incision pattern is confined to a limited area of the tissue graft. The limited area may be defined by arrangement of the cutting edges on the cutting surface, or a mask member may be provided to define the limited area. A mask member may have a solid area and one or more open areas that correspond to the limited area, wherein when the mask member is configured to be arranged between the cutting surface and the tissue graft, and wherein the solid area protects the tissue graft from the cutting edges.

In some embodiments, the limited area has a perimeter that reduces in width toward an end or edge portion of the limited area. In some embodiments, the limited area has an end portion with a predefined shape which may be selected from a group comprising but not limited to elliptical, oval, diamond, triangular arrowhead, square, and rectangular.

In some embodiments, the cutting surface is provided in a base plate with the cutting edges directed upward to incise at least part of the tissue graft by downward movement of the tissue graft onto the cutting surface. The base plate may further comprise an alignment feature for positioning the tissue graft before the apparatus is used to incise at least part of the tissue graft. In some embodiments, the cutting surface is removeable and/or interchangeable in the base plate.

Preferably, the incision pattern creates multiple interstices or fenestrations in the tissue graft which provide for expansion of at least part of the prepared graft. Typically, the incision pattern comprises a plurality of cutting edges arranged end to end and in two or more columns. The cutting edges may have a length of e.g. 5 to 9 mm, preferably 6 to 8 mm and more preferably 7 to 8 mm. Ends of neighbouring cutting edges in a column may be spaced by e.g. 1-4 mm, preferably 2-3 mm. The columns may be separated by e.g. 1-2 mm, preferably by about 1.3-1.8 mm such as e.g. 1.5 mm.

Preferably, the tissue is a fascia tissue such as, but not limited to, fascia lata and rectus sheath fascia. Ideally, the tissue graft is an autologous tissue graft. Preferably, the cutting edges are configured to incise the tissue in a longitudinal direction of connective fibres in the tissue graft. In some embodiments, the apparatus may be configured to provide variable incision patterns.

In some embodiments, the cutting edges are coloured for improved visibility relative to the tissue graft.

The apparatus may be configured to prepare a tissue graft of approximately 10 to 60 mm in width and approximately 60 to 140 mm in length. In some embodiments, the apparatus is configured to provide expansion of up to 2:1, preferably 1.5:1.

Viewed from another aspect, the present invention provides a kit for preparing a tissue graft, the kit comprising: (a) a cutting surface having a plurality of cutting edges arranged in an incision pattern, wherein the cutting surface is configured to prepare the graft by incising at least part of a tissue graft according to the incision pattern; (b) a bearing surface configured to be arranged in opposition to the cutting surface; and (c) a housing containing the cutting surface and the bearing surface; wherein in use, the tissue graft is arranged between the cutting surface and the bearing surface and the cutting surface incises the tissue graft when pressure is applied to one or both of the bearing surface and the cutting surface.

In some embodiments of the disclosure, the cutting edges are arranged on the cutting surface for preparation of the graft for use in surgical treatment of pelvic organ prolapse by transvaginal repair, wherein the cutting edges are arranged to incise only a middle portion of the graft. In other embodiments, the cutting edges are arranged on the cutting surface for preparation of the graft for use in surgical treatment of pelvic organ prolapse by sacral colpopexy, wherein the cutting edges are arranged to incise only an end portion of the graft. In yet other embodiments, the cutting edges are arranged on the cutting surface for preparation of the graft for use in surgical treatment of stress urinary incontinence by mid-urethral sling surgery, wherein the cutting edges are arranged to incise only a middle portion of the graft having a width less than 20 mm when harvested.

In some embodiments of the kit, the cutting surface is provided on a base plate, with the cutting edges directed upward to incise at least part of the tissue graft by downward movement of the tissue graft onto the cutting surface. In some embodiments, the housing comprises a tray with a base portion which provides the base plate. The bearing surface may be provided on a tool with a handle portion adapted to be gripped by a user when in use to apply pressure to incise the tissue graft, the tool being supplied in the kit. The tool may include a roller, with the roller providing the bearing surface. Alternatively, the tool may include a pressure pad, with the pressure pad providing the bearing surface. The pressure pad may be substantially flat or substantially convex.

The cutting surface may be provided on a cutting tool with a handle portion adapted to be gripped by a user when in use, to apply pressure to incise the tissue graft, the tool being supplied in the kit. The cutting tool may include a roller, with the roller providing the cutting surface. Alternatively, the cutting tool may include a die pad, with the die pad providing the cutting surface. The die pad may be substantially flat or substantially convex. The bearing surface may include an alignment feature for positioning the tissue graft before use of the cutting surface to prepare the tissue graft. In some embodiments of the kit, the housing comprises a tray with a base portion which provides the bearing surface.

In various embodiments of the kit, the cutting edges may be arranged end to end and in two or more columns. The cutting edges may have a length of 5 to 9 mm, preferably 6 to 8 mm and more preferably 7 to 8 mm. Ends of neighbouring cutting edges in a column may be spaced by 1-4 mm, preferably 2-3 mm. The columns may be separated by 1-2 mm, preferably by about 1.3-1.8 mm and more preferably 1.5 mm. Cutting edges in adjacent columns are ideally offset, with a cutting edge in one column aligned with a space between cutting edges of an adjacent column.

In some embodiments of the kit, the cutting edges are coloured for improved visibility relative to the tissue graft. Preferably the kit is configured to provide expansion the of the prepared tissue in a width dimension of up to 2:1, preferably 1.5:1 compared to the unprepared tissue.

Another aspect of the present disclosure provides method of performing surgical repair of pelvic organ prolapse or stress urinary incontinence, using a tissue graft prepared using the apparatus or the kit as disclosed herein.

In some embodiments, the method of performing surgical repair of pelvic organ prolapse using a tissue graft prepared using the apparatus or kit disclosed herein further comprises the steps of: cutting a V-shaped or U-shaped incision in an end portion of the tissue graft which has not been incised by the cutting edges, to form a flap; and folding the flap outward to provide an extension of the graft in a length dimension.

In some embodiments, a method of performing a surgical repair of pelvic organ prolapse by anterior compartment repair using a tissue graft prepared using the apparatus or kit disclosed herein further comprises the step of: forming an elongate incision in an end portion of the tissue graft which has not been incised by the cutting edges, to create a pair of elongate graft elements for attachment to supporting structures of the pelvis during the surgical repair.

Another aspect of the present disclosure provides a tissue graft prepared using the apparatus or kit disclosed herein.

It is to be understood each of the various aspects described herein may incorporate features, modifications and alternatives described in the context of one or more other aspects, such as but not limited to the various apparatus and kits, and tools. For efficiency, such features, modifications and alternatives have not been repetitiously disclosed for each and every aspect although one of skill in the art will appreciate that such combinations of features, modifications and alternatives disclosed for some aspects apply similarly for other aspects and are within the scope of and form part of the subject matter of this disclosure.

### BRIEF DESCRIPTION OF DRAWINGS

The present invention will now be described in greater detail with reference to the accompanying drawings. It is to be understood that the embodiments shown are examples only and are not to be taken as limiting the scope of the invention as defined in the claims appended hereto.

FIGS. 1A to 1C are schematic illustrations of a cutting surface according to various embodiments of the disclosure.

FIG. 2 is a schematic illustration showing cutting edges arranged to form an incision pattern according to an embodiment of the disclosure.

FIGS. 3A to 3C are illustrations showing various embodiments of a bearing surface for use with a cutting surface.

FIG. 4 shows an example of a cutting surface with a sample of tissue that has been incised by the cutting edges.

FIG. 5 shows the incised tissue from FIG. 4 removed from the cutting surface and expanded, showing the interstices which provide for expansion in the width dimension.

FIGS. 6A to 6C are show various embodiments of a cutting surface provided as part of a cutting tool.

FIGS. 7A to 7C are illustrations showing further embodiments of a cutting surface provided as part of a cutting tool.

FIGS. 8A to 8C are schematic illustrations showing harvested fascia lata tissue grafts in which a limited area 310 of the graft has been incised by the cutting edges. FIGS. 8B(i) and 8B(ii) show modifications in which the graft includes incisions forming flaps for extending the effective length of the graft.

FIG. 9 is a schematic illustration showing an incision pattern confined to an oval area located medially of the harvested graft tissue.

FIG. 10 is a schematic illustration showing an incision pattern with split graft for use in anterior repair.

FIGS. 11A to 11C show embodiments of a cutting surface provided in a base plate. FIG. 11B further shows a bearing surface in the form of a roller and FIG. 11C shows a bearing surface in the form of a convex surface on a bearing tool.

FIGS. 12A and 12B are schematic illustrations showing kits according to embodiments of the disclosure.

### DETAILED DESCRIPTION

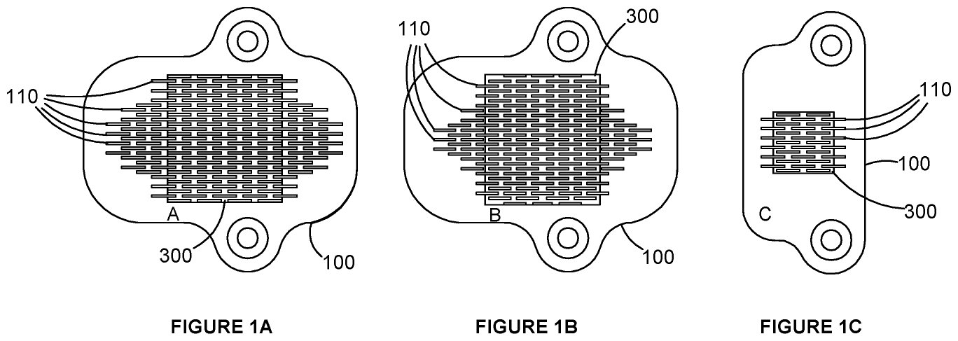

Referring firstly to FIGS. 1A to 1C, various embodiments of a cutting surface 100 for an apparatus of the present disclosure are presented in schematic form. Each cutting surface 100 has a plurality of cutting edges 110 arranged in an incision pattern and is configured to prepare a tissue graft 300 by incising at least part of the tissue according to the incision pattern. The incision pattern is configured to achieve expansion of at least part of the prepared graft in a width dimension.

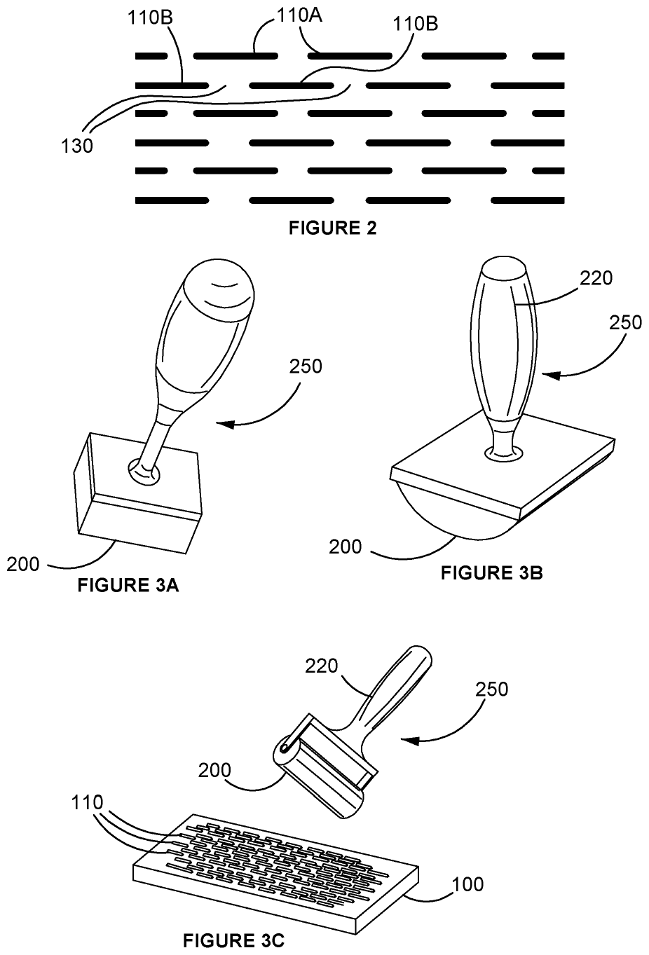

The cutting edges may be provided in various arrangements such as concentric circles which may have particular utility in hernia repair, parallel waves and the like. According to preferred embodiments, cutting edges 110 are arranged end to end and in two or more columns. As shown in the schematic illustration of FIG. 2, cutting edges 110A are offset from cutting edges 110B in an adjacent column so that a cutting edge 110A in one column is aligned with and preferably centred on a gap 130 between cutting edges 110B of an adjacent column. Various configurations may achieve an expansion ratio which is desirable for applications using fascia tissue, particularly those for which the tensile strength of the graft is important. For example an expansion ratio of approximately 1.5:1 may be achieved with cutting edges having a length of about 5 to 9 mm, preferably about 6 to 8 mm and more preferably about 7 to 8 mm, wherein ends of neighbouring cutting edges in a column are spaced by a gap length of about 1-4 mm, preferably about 2-3 mm, and wherein the columns are separated by about 1-2 mm, preferably by about 1.3-1.8 mm and more preferably, about 1.5 mm. For example a configuration employing cutting edges of approx. 7 mm length, a gap length of approx. 3 mm and column separation of approx. 2 mm achieves an expansion ratio of 1.5:1 but the integrity of the tissue may be diminished due to the tissue width between interstices when the tissue is expanded. A configuration employing cutting edges of approx. 6 mm length, a gap length of approx. 2 mm and column separation of approx. 2 mm also achieves an expansion ratio of less than 1.5:1 but may still be useful for applications that benefit from perforation but do not require much expansion. Meanwhile a preferred configuration employing cutting edges of approx. 7 mm length, a gap length of approx. 3 mm and column separation of approx. 1.5 mm achieves an expansion ratio of 1.5:1 while maintaining tissue integrity and uniform expansion of the tissue. Increasing the cutting edge to a length of approx. 8 mm also achieves good performance.

FIGS. 3A to 3C show various embodiments of a bearing surface 200 for use with a cutting surface 100 (such as those illustrated in FIGS. 1A to 1C) according to an apparatus of the present disclosure. Bearing surface 200 is configured to be positioned in opposition to the cutting surface 100 such that in use, the tissue graft is arranged between the cutting surface and the bearing surface, and the cutting surface incises the tissue graft when pressure is applied either through the cutting surface or the bearing surface, or both. In the examples illustrated in FIGS. 3A to 3C, bearing surface 200 is attached to a handle portion 220 forming a bearing tool 250 which enables the user to apply pressure through the bearing tool.

In use, the user arranges a harvested tissue graft 300 on cutting surface 100, taking care not to snag or damage the tissue on the cutting edges 110. Ideally, the harvested tissue graft is fascia tissue such as fascia lata or rectus sheath fascia and the user arranges tissue 300 with the fibres running parallel with cutting edges 110. The user grasps the handle portion 220 of tool 250, positions the bearing surface 200 over the tissue, and applies pressure through the handle so that the cutting edges 110 incise the tissue 300. The bearing surface 200 may be substantially flat as shown in FIG. 3A, or it may be convex as shown in FIG. 3B. A convex surface may enable more even pressure to be applied to all cutting edges 110 by the user rocking the bearing surface 200 back and forth while applying pressure. In another arrangement illustrated in FIG. 3C, the bearing surface 200 is provided in the form of a roller drum and pressure is applied by the user grasping handle portion 220 rolling the roller drum over the tissue and cutting surface 100. Ideally the roller (and indeed the convex bearing surface of FIG. 3B) is rolled along the longitudinal dimension of the cutting edges 110 to produce clean incisions, maintain the integrity of the tissue and preserve tensile strength in the longitudinal direction.

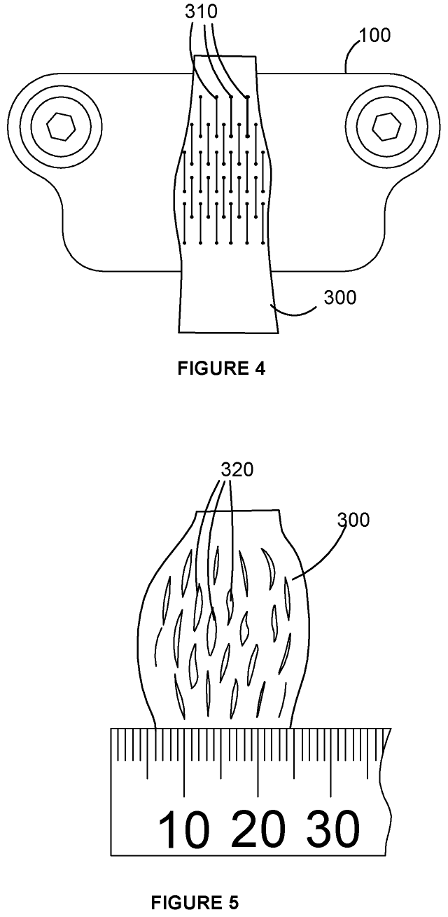

FIG. 4 shows an example of a cutting surface 100 formed substantially according to the illustration in FIG. 1C, with a harvested tissue graft 300 that has been prepared using the apparatus. Cutting edges 110 are very fine and have formed incisions 310 in the tissue 300 through which the cutting edges protrude. In some embodiments, the cutting edges 110 or a portion of them may be coloured so that the user can inspect the tissue 300 and confirm that all cutting edges have fully penetrated and incised the graft. Ideally a contrasting colour to the fascia lata or other graft tissue, is utilised, such as blue, preferably royal blue, or green, such as blue green or lime green. FIG. 5 shows the prepared tissue 300 removed from the cutting surface 100 and expanded, clearly showing the interstices 320 which provide for expansion in the width dimension (i.e. in a lateral direction relative to the longitudinal dimension of the incisions formed by the cutting edges) by a factor of approximately 1.5 from approximately 15 mm to approximately 22 mm where the incisions have been placed. Advantageously, the apparatus does not require the entire tissue graft 300 to be incised according to the incision pattern. Rather, only the part of the tissue graft 300 that is required to be expanded is prepared by the formation of incisions in the incision pattern, and the remaining parts of the tissue graft remain fully intact. Examples of surgical applications where it is desirable to expand only part of the tissue graft follow.

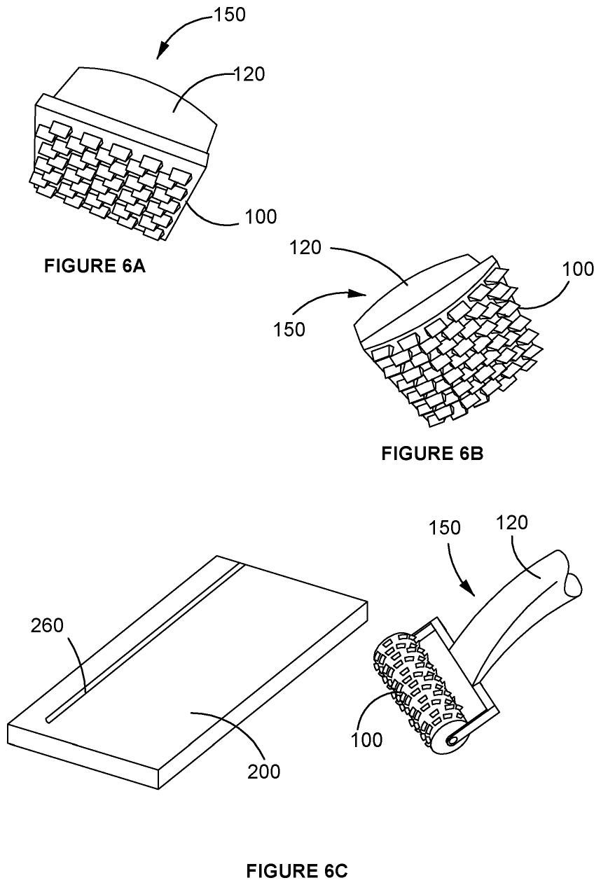

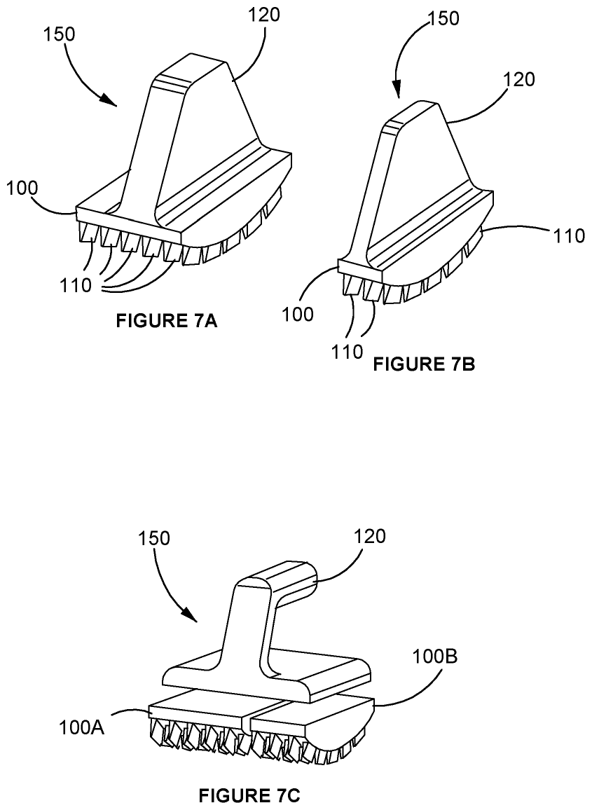

FIGS. 1A to 4 show embodiments in which the cutting surface 100 is a substantially flat surface provided in a base plate with the cutting edges 110 directed upward to incise at least part of the tissue graft by downward movement of the tissue graft onto the cutting surface. However that need not be the case and in other arrangements, the bearing surface 200 may be provided in or as a base plate such as a silicon, rubber, plastic, ceramic or metallic mat, for example, with the cutting edges 110 provided on a cutting tool. Examples of such cutting tools are provided in FIGS. 6A to 7B.

In FIG. 6A, cutting surface 100 is substantially flat, whereas in FIG. 6B, the cutting surface 100 is convex. In both examples, a handle portion 120 is provided to form a cutting tool 150. In use, the user arranges the tissue 300 on the bearing surface (not shown). In use, the user grasps the handle portion 120 of cutting tool 150 and positions the cutting surface 100 over the tissue 300 and the bearing surface, aligning the cutting edges 110 of the cutting surface 100 to be parallel with the fibres of the tissue. The user applies pressure through the handle portion 120 so that the cutting edges 110 incise the tissue. A convex cutting surface, as in FIG. 6B, may provide a superior result as the convex shape allows the user to rock the cutting surface back and forth while applying pressure. For the cutting surfaces in FIGS. 6A and 6B, a side-to-side scrubbing action may be applied to ensure that the cutting edges 110 have penetrated the full thickness of the tissue. FIG. 6C, shows the cutting surface 100 in the form of a roller and pressure is applied by the user grasping handle portion 120 and rolling the cutting surface over the tissue and the bearing surface 200. Ideally, the roller (and indeed the convex cutting surface of FIG. 6B) is rolled with the cutting edges aligned along the longitudinal dimension of the fibres in the tissue to form clean incisions, maintain the integrity of the tissue and preserve tensile strength in the longitudinal direction.

The bearing surface 200 of FIG. 6C also provides an alignment feature 260. In use, the user may position the harvested tissue graft with the fibres aligned with the alignment feature, and use the alignment feature to guide application of the cutting surface to so that the cutting edges form incisions in the longitudinal direction of the fibres. Alternatively/additionally, the bearing surface 200 may be a mat, such as a silicon or rubber mat, that is configured for use with one or more pins to aid in positioning or “pegging out” the harvested tissue graft prior to application of the cutting surface. In yet other embodiments, the bearing surface 200 may have one or more pegs or protrusions arranged to retain a suture loop that has been pre-placed on the harvested tissue graft. Preferably the suture loop is further utilised during placement of the prepared graft during surgery.

The convex cutting surfaces of FIGS. 6B and 6C show the cutting edges arranged 110 arranged orthogonally to an axis around which the curvature of the cutting surface rotates. However, it is to be understood that in an alternative arrangement, the cutting edges could be arranged parallel to an axis around which the cutting surface rotates. This would require the user to align the cutting edges with the longitudinal direction of fibres in the harvested tissue graft before applying pressure, and rocking/rolling the cutting tool across columns of cutting edges, rather than along them.

FIGS. 7A and 7C provide further examples of a cutting tool 150 having a cutting surface 100 with cutting edges 110 coupled with a handle portion 120. It is to be understood that the number of cutting edges provided in the cutting surface may be determined according to the procedure for which the tissue graft is being prepared, as may the incision pattern formed by the cutting edges or the areas of the tissue graft that are to be incised. In FIG. 7A cutting tool 150 provides 5 columns of cutting edges 110, whereas the embodiment of FIG. 7B provides only 2 columns of cutting edges. The embodiment of FIG. 7C shows an arrangement in which two cutting surfaces 100A and 100B having 5 and 3 columns of cutting edges respectively, are combined by handle portion 120 into a single cutting tool 150 suitable for preparation of a wider tissue graft by use of a total of 8 columns of cutting edges. This arrangement provides flexibility in the number of columns of cutting edges, and the area of the tissue to be incised. The cutting surfaces 100A and 100B may couple with handle portion 120 by any suitable means such as magnetic or friction fit couplings for the like, as would be appreciated by one of skill in the art.

Advantageously, embodiments of the invention provide for the incision pattern formed in the tissue by the cutting edges to be confined to a limited area of the tissue graft. This is not possible with existing skin meshers which mesh the entirety of the tissue graft and do not provide for meshing of only a portion of the graft as may be required for some procedures. In some embodiments, the apparatus may further comprise a mask member for limiting the area of incised tissue. The mask member may comprise a solid area and one or more open areas that correspond to the limited area to be incised. In use, the mask member is arranged between the cutting surface and the tissue graft and when pressure is applied, the solid area of the mask member protects the tissue graft from the cutting edges such that the cutting edges penetrate only the unmasked area of the tissue.

In other embodiments, the limited area of the tissue graft that is incised is defined by extent, or limited area, of the cutting edges on the cutting surface. For example, in the case of FIGS. 1A to 1C, the incision patterns formed by cutting edges 110 are confined to a limited area of the tissue graft making them suitable for preparation of a fascia lata graft for sacral colpopexy, trans-vaginal repair and mid-urethral sling surgery respectively. In this example, the cutting edges 110 are 7 mm long and arranged in columns with cutting edges spaced 3 mm apart end to end, with column centres spaced 1.5 mm apart. The tissue samples 300 provided in these figures is illustrative only, to demonstrate visibility of the cutting edges 110 after they have incised the tissue 300. In the clinical setting, fascia tissue will be harvested in sizes and dimensions selected according to the clinical application as will be described in the examples below. Advantageously, embodiments of the present disclosure may be utilised for preparation of relatively small grafts, such as approx. 10 to 60 mm in width and approx. 60 to 140 mm in length.

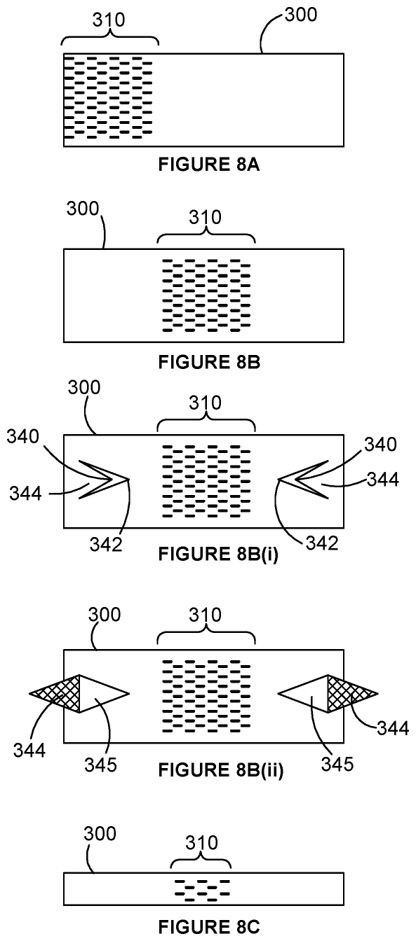

FIGS. 8A to 8C are schematic illustrations representing harvested tissue grafts 300 prepared according to embodiments of the disclosure, in which a limited area 310 of the graft has been incised by the cutting edges. FIG. 8A represents a tissue graft 300 approximately 120 mm long and approximately 30 mm wide which may be suitable for treatment of POP by sacral colpopexy. In this case the incisions formed by the cutting edges modify only an end portion 310, such as an end third, of the graft. For this indication, the modified section 310, when expanded, need only be approximately 40 mm wide which is achieved by the present disclosure which, in some embodiments, provides an expansion ratio of 1.5:1.

FIG. 8B represents a tissue graft 300 approximately 120 mm long and approximately 30-40 mm wide which may be suitable for treatment of POP by anterior repair. In this case, the incisions formed by the cutting edges modify a central portion 310, such as a central third, of the graft. For this indication the modified section 310, when expanded, need only be approximately 40-60 mm wide. While the incisions may be formed to the edge of the graft, in some embodiments it may be desirable for the incision pattern to allow for a border of un-incised tissue to allow the edges to be sutured. FIG. 8B(i) provides a modification of the graft in FIG. 8B, in which incisions 340 are formed in side portions of the harvested tissue graft 300. The incisions 340 ideally form a V-shape or U-shape with the apex 342 normally directed toward the incised area 310. The incisions 340 may be made using a scalpel or a cutting tool similar to the cutting tool described herein but with a V- or U-shaped cutting edge instead of cutting edges arranged to form an incision pattern for expansion of the graft. Incisions 340 create a flap 344 which can be folded out as shown in FIG. 8B(ii), to extend the length of the graft, if required. Void 345 remains after the flap 344 is outwardly folded. One of skill in the art would appreciate that the shaded portion on flap 344 designates the underside of the graft tissue. Although two incisions 340 and extension flaps 344 are shown in FIGS. 8B(i) and 8B(ii), it is to be understood that only one such incision and flap may be required to achieve the required extension. Advantageously, use of extension incisions 340 and flaps 344 provide further mechanisms for minimising tissue harvested from the donor site and can allow for extension of the graft length of up to approx. 30%.

FIG. 8C represents a tissue graft of approximately 100 mm long and 11 mm width which may be suitable for treatment of SUI by mid-urethral sling surgery. In this case the incisions formed by the cutting edges modify only a middle section 310 of the graft. For this indication, the incisions are beneficial for alleviating or mitigating tissue retraction and shrinkage of the graft. Shrinkage or retraction of the sub-urethral portion of the graft may increase the risk of post-operative voiding difficulty (inability to effectively empty the bladder of urine), an unwanted complication of surgery.

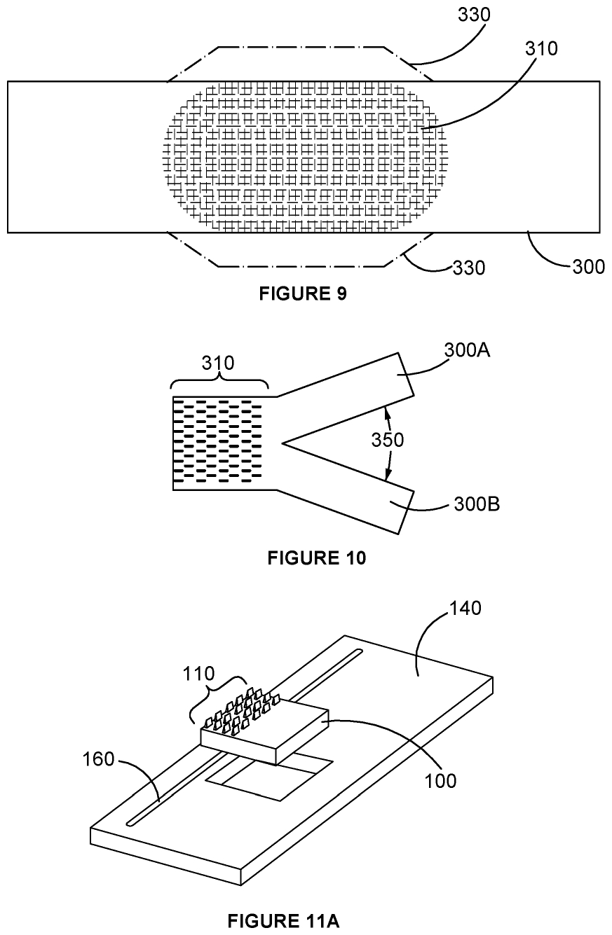

The schematic illustrations of FIGS. 8A to 8C show square and rectangular sections of modified tissue 310. However, it may be desirable that the end portions of the modified sections 310 graduated for a smoother transition between expanded and unexpanded portions of the graft 300. This may be achieved by a mask member or an incision pattern in which the limited area 310 is shaped to reduce in width toward an end of the limited area. One example is provided in the schematic illustration of FIG. 9 which shows an oval area 310 in which the incision pattern is formed. In this example, the broken lines 330 represent the shape of the modified tissue graft 310 when expanded. Other end shapes that could lend themselves to a smoother transition between unexpanded and expanded portions of the tissue graft include elliptical, triangular, diamond or arrowhead ends, to name a few. Cutting edges 110 toward the perimeter of the limited area may be shortened in order to provide contoured end shapes for the incision pattern. Alternatively, the end shapes of the limited area may approximate an oval, semicircle, ellipse, triangle, diamond, arrowhead or the like only insofar as the arrangement of the full length cutting edges allow (see for example the graduated perimeters of FIGS. 1A and 1B).

FIG. 10 is a schematic illustration of yet another modified tissue graft suitable for treatment of POP by anterior repair. In this configuration, a tissue graft 300 approximately 120 mm long and approximately 30-40 mm wide. The incisions formed by the cutting edges modify an end portion 310, such as a third to up to half of the graft length. An elongate incision 350 splits the opposing end portion, such as a third to up to half of the graft length, into two elongate graft elements 300A, 300B. This configuration provides an alternative graft for anterior vaginal compartment repair in which there is the option to deploy the graft in a different orientation e.g. having up to 90 degrees rotation, compared with the prepared grafts of FIGS. 8B, 8B(i), 8B(ii) and FIG. 9. Although not shown, it is to be appreciated that the end portions of modified section 310 may be graduated for a smoother transition between the expanded portion of the graft and particularly that portion of the graft having split elements 300A, 300B.

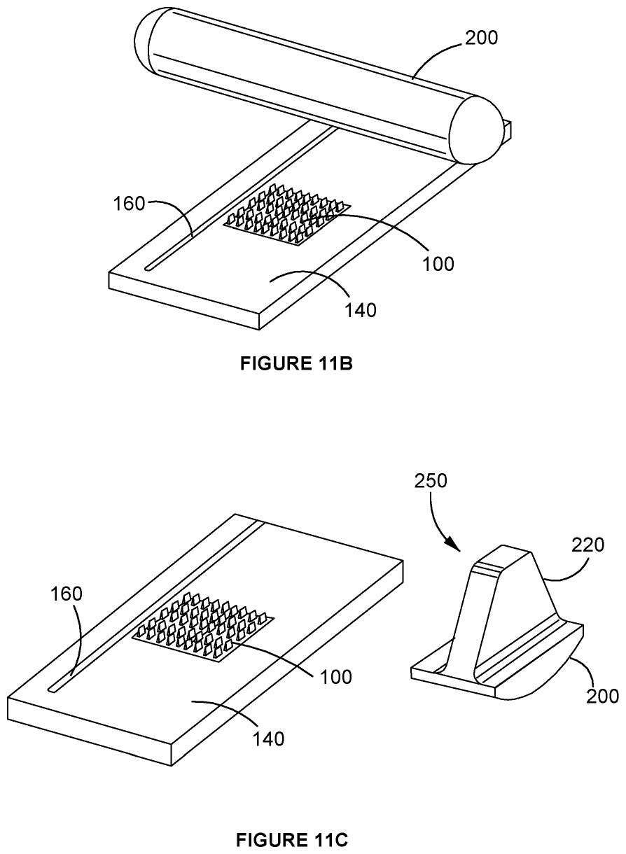

FIGS. 11A to 11C show variations on the embodiments shown in FIGS. 1A to 4, in which the cutting surface 100 is a substantially flat surface provided in a base plate 140 with the cutting edges 110 directed upward to incise at least part of the tissue graft by downward movement of the tissue graft onto the cutting surface. While these figures show the cutting edges 110 arranged in a rectangular or square incision pattern, it is understood that this need not be the case, and various shapes may be provided, such as those with graduated end portions, as described above. FIG. 11A illustrates an example in which cutting surface 100 is removable from base plate 140. This may be desirable in embodiments where the base plate is intended for re-use after cleaning and sterilisation, but wherein the cutting surface may be disposed of between patients, reducing the risk of transfer of biohazard and diminished performance of the apparatus by degradation of the cutting edges 110 with use. This arrangement may reduce the extent of single use components thereby reducing medical waste attributable to preparation of a tissue graft using this embodiment of the invention.

Also shown in FIGS. 11A to 11C is an alignment feature 160 which may be used to position the harvested tissue graft on the cutting surface before the bearing surface is applied to form the incisions in the tissue. It is to be understood, however, that the cutting edges 110 themselves may be used as an alignment guide for correct placement of the tissue on the cutting surface, i.e. with the fibres of the tissue graft running parallel with the cutting edges, and hence the alignment feature may be omitted. FIGS. 11B and 11C also show examples of bearing surfaces through which pressure may be applied to the tissue graft arranged on the cutting surface 100 to from incisions in the incision pattern. In FIG. 11B the bearing surface 200 is in the form of a roller which may be rolled over the tissue and the cutting surfaces. In other examples, the roller may be coupled with a handle portion to form a bearing tool, as in FIG. 3C. In FIG. 11C, a bearing tool 250 comprises a convex surface 200 with a handle portion 220.

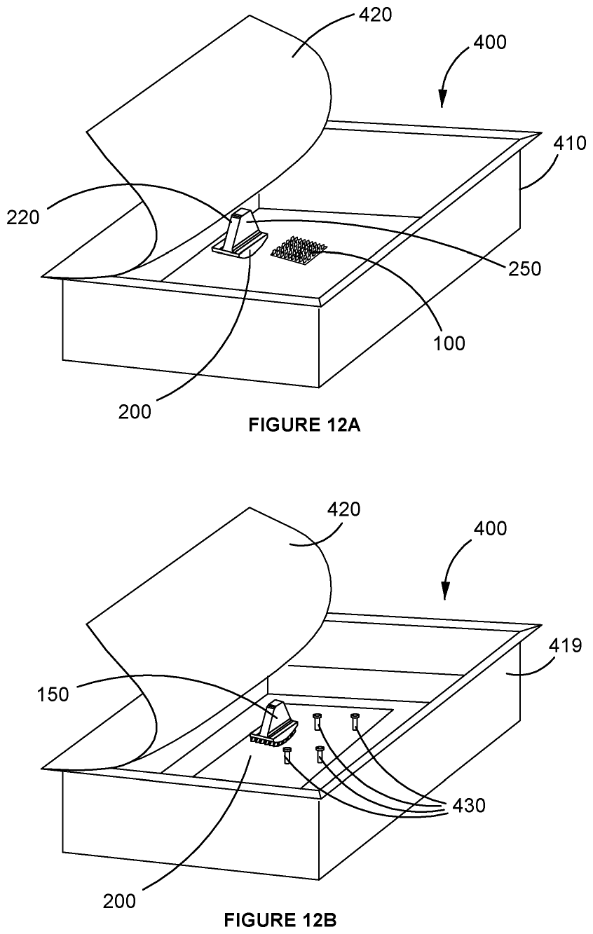

To facilitate the process of preparing a tissue graft according to embodiments of the disclosure it may be desirable to provide various component parts of the apparatus in kit form. Such a kit 400 (FIG. 12A) may comprise a housing 410 which contains a cutting surface 100 and a bearing surface 200 according to the various embodiments herein. In some embodiments, it may be desirable for the kit to be configured for particular surgical procedures. For example, a kit may be provided for preparation of a graft for use in surgical treatment of pelvic organ prolapse by transvaginal repair, wherein the cutting edges 110 are arranged to incise only a middle portion of the graft. In another example, a kit may be provided for preparation of a graft for use in surgical treatment of pelvic organ prolapse by sacral colpopexy, wherein the cutting edges 110 are arranged to incise only an end portion of the graft. In another example, a kit may be provided for preparation of a graft for use in surgical treatment of stress urinary incontinence by mid-urethral sling surgery, wherein the cutting edges 110 are arranged to incise only a middle portion of the graft and wherein the graft has a width less than 20 mm.

In some arrangements, the cutting surface of the kit is provided on a base plate 140, with the cutting edges 110 directed upward to incise at least part of the tissue graft by downward movement of the tissue graft onto the cutting surface akin to the examples presented in FIGS. 1A to 1C and 11A to 11C. Preferably, the cutting edges 110 are arranged on the base plate 140 according to the surgical treatment for which the kit is provided. Ideally the kit further provides a bearing tool 250 providing a bearing surface 200 with a handle portion 220 adapted to be gripped by a user, to apply pressure to incise the tissue graft. The bearing tool 250 may comprise a roller or a flat or convex pressure pad providing a bearing surface 200.

The housing may include a tray with a removable plastic seal 420 to contain other sterile contents of the kit, and/or a simple sterile wrap or packaging (not shown) which, in some embodiments, may be utilised also to facilitate disposal of the apparatus after use. In arrangements where the housing comprises a tray 410, the tray itself may form the base plate 140 from which the cutting edges 110 upwardly extend, with the cutting edges being located in the tray such that they allow for arrangement of the harvested tissue graft and incisions to be formed only in the part of the graft which is required for the specific surgical procedure. In other embodiments, the base plate 140 is separate from and contained in the tray when the kit is supplied.

In other embodiments, the kit 400 (FIG. 12B) includes a cutting tool 150 having a cutting surface 100 attached to a handle portion 120 adapted to be gripped by a user when in use, to apply pressure to incise the tissue graft. The cutting tool 120 may include a cutting surface 100 incorporated into a roller or a die pad which may be flat or convex. The bearing surface 200 of the kit may be provided as a silicon, rubber, plastic, ceramic or metallic mat or base plate supplied as a part inside kit 400 or incorporated into the base of the kit housing 410. The bearing surface 200 and may provide one or more alignment features for positioning the harvested tissue graft before use of the cutting surface to prepare the tissue graft. Such alignment features may be built into the bearing surface 200 and/or the kit may contain one or more pins 430 for pinning or “pegging out” the harvested tissue graft onto the bearing surface. In some embodiments, the bearing surface may have one or more protrusions arranged to retain a suture loop that has been pre-placed on the harvested tissue graft, to aid in positioning of the graft on the bearing surface.

Each of the cutting surface 100, the bearing surface 200 and the handle portion 120, 220 (where provided) when provided in kit 400 or separate component parts, may be manufactured from any suitable material such as plastics, polymers, stainless or other medical grade steel, ceramics or other materials that provide for cutting edges that are sufficiently sharp to incise the tissue graft. The cutting edges may be moulded, embedded or fabricated into the cutting surface which may be flat or curved. The present disclosure is primarily directed to preparation of a fascia tissue graft such as fascia lata or rectus sheath fascia, which is thin but fibrous. Thus the cutting edges must completely and cleanly incise the tissue to avoid damage and degradation of the graft.

In some embodiments, parts or all of the apparatus may be re-useable after cleaning and sterilisation. Alternatively, parts may be interchangeable and replaceable. For instance, a cutting tool 150 with a handle portion 120 may have a cutting surface 100 which is removable and replaceable with a new cutting surface. The materials from which these component parts are manufactured may differ according to their function. However in some circumstances it may be preferred that the apparatus is a single use device to avoid risk of transfer of biohazard. In some embodiments it may be desirable for the apparatus to be manufactured from a single type of material so that it may be recycled following use. Thus, in some embodiments a cutting tool 150 or bearing tool 250 comprising a handle and cutting/bearing surface may be formed as a unitary material product. Suitable materials may include e.g. stainless steel or other medical grade steels, ceramics or recyclable polymeric materials that can be manufactured with sufficient hardness to meet the required cutting performance of the cutting edges.

### Example 1: Harvesting and Preparation of Autologous Fascia Lata

For pelvic organ prolapse surgery a 12×4 cm strip of fascia lata is harvested from either the right or left lateral thigh through a single skin incision. For stress urinary incontinence surgery 10×1 cm strip of fascia lata is harvested from either the right or left thigh through a single skin incision.

For transvaginal surgery to treat an anterior or posterior vaginal wall prolapse, the central portion of the graft is incised using apparatus disclosed herein. This increases the central width of the graft from 4 cm to 6 cm (FIGS. 1B, 8B). Alternatively, the graft of FIG. 10 may be utilised in anterior repair, where an end portion of the graft is prepared using the cutting edges as disclosed herein, with an opposing end portion of the graft cut to form a pair of elongate graft elements for attachment to supporting structures of the pelvis. For sacral colpopexy to treat vaginal apical prolapse, the distal portion of the graft is incised using the POP graft latticing device. This increases the distal width of the graft from 4 cm to 6 cm (FIGS. 1A, 8A). For surgery to treat stress urinary incontinence, the central portion of the graft is incised using the SUI graft latticing device. This increases the width of the graft from 1 cm to 1.5 cm (FIGS. 1C and 8C).

While the dimensions of fascia lata graft provided in the examples are preferred, it is to be understood that the fascia lata graft dimensions may vary depending on an individual patient's clinical findings.

### Example 2: Transvaginal Repair Using Expanded Autologous Fascia Lata

When performing surgery to treat the anterior vaginal compartment with or without concomitant apical prolapse an incision is made in the anterior vaginal wall epithelium. The epithelium is dissected off the underlying prevesical tissue. Dissection is continued to the vaginal apex, bladder neck and towards the pelvic sidewalls on each side. Depending on the specific anatomic defects, dissection is undertaken to create channels to each sacrospinous ligament or to the parietal fascia of the obturator internus muscle anterior to the ischial spine. The prevesical tissue is repaired using a colporrhaphy technique. The centrally incised autologous fascia lata graft is introduced into the anterior vaginal compartment where it is expanded to the desired width when it is placed into the target anatomic position. To achieve this position, the lateral straps are placed into the right and left channels (either onto each sacrospinous ligament or onto the parietal fascia of the obturator internus muscle). In selected cases, the straps of the fascia lata graft are sutured onto the left and right sacrospinous ligaments. Alternatively, anterior repair may utilise a fascia lata graft prepared with expansion incisions formed over an end portion of the graft tissue and an elongate incision dividing the opposing end into a pair of elongate tissue elements (see FIG. 10). To achieve placement of this graft the elongate elements are attached to sacrospinous ligaments or placed onto parietal fascia of obturator internus muscle with the orientation of the graft having the appearance of upside down trousers compared to the hammock-like arrangement of centrally incised grafts utilised in anterior repair. V-shaped or U-shaped incisions may be placed in one or both end portions of the lateral straps of the graft to provide an extension flap in circumstances where extra graft length is required. The extension flap is then folded out from the graft and tacked to the sacrospinous ligaments. The central portion of the graft is tacked with sutures to cover the anterior vaginal wall defect. The vaginal epithelium is trimmed if required and closed with sutures. At the completion of the anterior vaginal compartment surgery it is recommended that a cystoscopy is undertaken to exclude any lower urinary tract injury.

When performing surgery to treat the posterior vaginal compartment with or without concomitant apical prolapse an incision is made in the posterior vaginal wall epithelium. The epithelium is dissected off the underlying pre-rectal tissue. Dissection is continued to the vaginal apex and towards the pelvic sidewalls on each side. Dissection is undertaken to create channels onto each sacrospinous ligament. The pre-rectal tissue is repaired using a colporrhaphy technique. The centrally incised autologous fascia lata graft is introduced into the posterior vaginal compartment where it is expanded to the desired width when it is placed into the target anatomic position. To achieve this position, the lateral straps are placed into the right and left sacrospinous ligaments, utilising extension flaps formed by V-shaped or U-shaped incisions, if required. In selected cases, the straps of the fascia lata graft are sutured onto the left and right sacrospinous ligaments. The central portion of the graft is tacked with sutures to cover the posterior vaginal wall defect. The vaginal epithelium is trimmed if required and closed with sutures. At the completion of posterior vaginal compartment surgery and anterior compartment surgery when sacrospinous sutures are placed, it is recommended that a digital rectal examination is undertaken to exclude any rectal injury.

At the completion of transvaginal surgery using expanded autologous fascia lata, a supporting splint is placed into the lumen of the vagina and sutured into position for 3-4 weeks. The main purpose of the splint is to support the position of the graft during its incorporation into the patient's tissue in the post-operative period and to reduce the risk of the graft becoming detached from the points of graft attachment.

### Example 3: Sacral Colpopexy Using Expanded Autologous Fascia Lata

Sacral colpopexy may be undertaken by laparoscopy, robotic-assisted laparoscopy, or laparotomy. Depending on the anatomic defects, the bladder is reflected off the upper anterior vagina and the rectum is reflected off the posterior vagina. The peritoneum is dissected from sacral promontory to cul-de-sac on the right of the rectum and sigmoid and below the right ureter. The expanded autologous fascia lata graft is introduced into the pelvis and the expanded portion of the graft is attached to the vaginal apex and either upper anterior or posterior vaginal walls and to the anterior longitudinal ligament on the sacral promontory. The peritoneum is then closed over the graft. At the completion of surgery it is recommended that a cystoscopy is undertaken to exclude any lower urinary tract injury. A digital rectal examination should be undertaken to exclude rectal injury. At the completion of the sacral colpopexy using expanded autologous fascia lata, a supporting splint is placed into the lumen of the vagina and sutured into position for 3-4 weeks.

### Example 4: Mid-Urethral Sling Using Expanded Autologous Fascia Lata

Surgery commences with a sub-urethral vaginal incision. With retropubic placement of the sling, tunnels are created towards the perineal membrane on each side of the urethra. Suprapubic skin incisions (approximately 5 mm in length) are made. Sutures are attached to each end of the autologous fascia lata sling. Using a retropubic sling placement needle, the sutures attached to the ends of the sling straps are passed from the vaginal incision through the channels created to the perineal membrane on each side, into the retropubic space behind the pubic bone on each side and through the anterior abdominal wall incisions. The sub-urethral expanded portion of the sling is sutured to the pre-urethral tissue. The sling is positioned without tension. The sutures are cut flush with the skin incisions. Cystoscopy is undertaken to exclude any lower urinary tract injury. The vaginal and skin incisions are closed.

With a trans-obturator placement of the sling, tunnels are created towards the obturator membrane on each side with care taken to avoid the obturator fossa on each side. Incisions (approximately 5 mm in length) in the groin skin are made at the level of the external urinary meatus. Sutures are attached to each end of the autologous fascia lata sling. Using a trans-obturator sling placement needle, the sutures attached to the ends of the sling straps are passed from the vaginal incision through the channels created to the obturator membrane on each side, through the obturator membrane remote from the obturator fossa on each side and then through the skin incisions in the groin. The sub-urethral expanded portion of the sling is sutured to the pre-urethral tissue. The sling is positioned without tension. The sutures are cut flush with the skin incisions. Cystoscopy is undertaken to exclude any lowly urinary tract injury. The vaginal and skin incisions are closed.

Embodiments of the present disclosure facilitate expansion of autologous fascia lata to transform into a “mesh” or a mesh section. This provides for a true biological mesh that allows for its use where synthetic and non-autologous biological meshes have been previously used. For the same reasons that synthetic mesh rather than non-meshed sheets is used, the use of fascia lata without expansion in these applications not only requires larger amounts of fascia lata to be harvested, it could cause seromas and reduce potential for optimal tissue integration of the graft.

Tools specifically designed for preparation of tissue according to embodiments of the present disclosure provide for expansion of the width dimension of the harvested autologous fascia lata by a factor of approximately 1.5 to up to 2. For example, the present disclosure provides for preparation of a tissue graft (e.g. fascia lata) for the treatment of POP which results in a graft that is sufficiently wide at the incised section to cover the anatomic defect without needing to harvest a graft greater than 40 mm in width. This allows the autologous fascia lata graft to be harvested through one relatively small incision in the lateral thigh resulting in less donor site morbidity when compared to taking a much wider (e.g. 60 mm) strip of fascia lata as is required when used in the harvested, unprepared form. The expanded autologous fascia lata results in interstices within the body of the graft which may enhance tissue ingrowth into the interstices of the graft which in turn may reduce graft retraction, graft-related chronic inflammation, and seroma formation. Tissue grafts prepared according to embodiments of the disclosure provide a safe and effective alternative to synthetic mesh, xenografts and allografts for treatment of e.g. POP and SUI and advantageously, can be deployed using known surgical methods.

While embodiments of the present disclosure are described for use in pelvic floor surgery, specifically prolapse and incontinence surgery, it is to be understood that grafts prepared according to the present disclosure could also be utilised in treatment of incisional, congenital and acquired herniae, facial reconstructive surgery, breast reconstruction and used as an adhesion barrier. Further, while embodiments of the disclosure are described for an autologous tissue graft, it is to be understood that these embodiments have similar utility in the preparation of xenograft and allograft tissue as well as cadaver tissue.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components or group thereof.

It is to be understood that various modifications, additions and/or alterations may be made to the parts previously described without departing from the ambit of the present invention as defined in the claims appended hereto.

Future patent applications may be filed on the basis of the present application. It is to be understood that the following claims are provided by way of example only, and are not intended to limit the scope of what may be claimed in any such future application. Features may be added to or omitted from the claims at a later date so as to further define or re-define the invention or inventions.

1-53. (canceled)

54. Apparatus for preparing a tissue graft, the apparatus comprising a cutting surface having a plurality of cutting edges arranged in an incision pattern, wherein the cutting surface is configured to prepare the tissue graft by incising at least part of the tissue graft according to the incision pattern, said incision pattern being configured to achieve expansion of at least part of the prepared graft.

55. The apparatus of claim 54, further comprising a bearing surface configured to be arranged in opposition to the cutting surface, wherein in use, the tissue graft is arranged between the cutting surface and the bearing surface and the cutting surface incises the tissue graft when pressure is applied to one or both of the bearing surface and the cutting surface.

56. The apparatus of claim 55, wherein one of the cutting surface and the bearing surface is a substantially convex surface and optionally wherein the other of the cutting surface and the bearing surface is a substantially flat surface; and further optionally wherein the convex surface is a roller.

57. The apparatus of claim 54, wherein the cutting surface is a substantially flat surface, or wherein the cutting surface is a substantially convex surface.

58. The apparatus of claim 54, wherein the cutting surface is provided on a die pad with a handle portion adapted to be gripped by a user to apply pressure to incise the tissue graft, and optionally wherein two or more of the cutting surface, the die pad and the handle portion are formed from a unitary piece of material.

59. The apparatus of claim 54, wherein the incision pattern is confined to a limited area of the tissue graft, and optionally:
wherein the limited area is defined by arrangement of the cutting edges on the cutting surface; and/or
wherein the limited area has an end portion with a predefined shape and optionally wherein the predefined shape is selected from a group comprising but not limited to elliptical, oval, diamond, triangular, arrowhead, square and rectangular; and/or
wherein the limited area has a perimeter reducing in width toward an end of the limited area.

60. The apparatus of claim 59, further comprising a mask member, the mask member having a solid area and one or more open areas that correspond to the limited area, wherein when the mask member is configured to be arranged between the cutting surface and the tissue graft, and wherein the solid area protects the tissue graft from the cutting edges.

61. The apparatus of claim 54, wherein the cutting surface is provided in a base plate with the cutting edges directed upward to incise at least part of the tissue graft by downward movement of the tissue graft onto the cutting surface, and optionally wherein the base plate further comprises an alignment feature for positioning the tissue graft before the apparatus is used to incise at least part of the tissue graft; and/or wherein the cutting surface is removeable and/or interchangeable in the base plate.

62. The apparatus of claim 54, wherein the incision pattern creates multiple interstices in the tissue graft which provide for expansion of at least part of the prepared graft, and optionally wherein the cutting edges are configured to incise the tissue in a longitudinal direction of connective fibres in the tissue graft.

63. The apparatus of claim 54, wherein the tissue is a fascia tissue such as but not limited to fascia lata and rectus sheath fascia.

64. The apparatus of claim 54, wherein the incision pattern comprises a plurality of cutting edges arranged end to end and in two or more columns; and optionally there is one or more of:
wherein the cutting edges have a length of about 5 to 9 mm, preferably about 6 to 8 mm and more preferably about 7 to 8 mm;
wherein ends of neighbouring cutting edges in a column are spaced by about 1-4 mm, preferably about 2-3 mm; and
wherein the columns are separated by about 1-2 mm, preferably by about 1.5-2 mm.

65. The apparatus claim 54, configured to prepare a tissue graft of approximately 10 to 60 mm in width and approximately 60 to 140 mm in length; and/or wherein the apparatus is configured to provide expansion ratio of up to 2:1, preferably 1.5:1.

66. A tissue graft prepared using the apparatus according to claim 54.

67. A kit for preparing a tissue graft, the kit comprising:
(a) a cutting surface having a plurality of cutting edges arranged in an incision pattern, wherein the cutting surface is configured to prepare the graft by incising at least part of a tissue graft according to the incision pattern;
(b) a bearing surface configured to be arranged in opposition to the cutting surface; and
(c) a housing containing the cutting surface and the bearing surface;
wherein in use, the tissue graft is arranged between the cutting surface and the bearing surface and the cutting surface incises the tissue graft when pressure is applied to one or both of the bearing surface and the cutting surface.

68. The kit according to claim 67, wherein the cutting edges are arranged on the cutting surface for preparation of the graft for use in one of:
surgical treatment of pelvic organ prolapse by transvaginal repair, wherein the cutting edges are arranged to incise only a middle portion of the graft;
surgical treatment of pelvic organ prolapse by sacral colpopexy, wherein the cutting edges are arranged to incise only an end portion of the graft; and
surgical treatment of stress urinary incontinence by mid-urethral sling surgery, wherein the cutting edges are arranged to incise only a middle portion of the graft having a width less than 20 mm when harvested.

69. The kit according to claim 67, further comprising an apparatus for preparing the graft, the apparatus including at least the cutting surface.

70. The kit according to claim 67, wherein the housing comprises a tray with a base portion which provides a base plate; or wherein the housing comprises a tray with a base portion which provides the bearing surface.

71. The kit according to claim 67, wherein the cutting surface is provided on a cutting tool with a handle portion adapted to be gripped by a user when in use, to apply pressure to incise the tissue graft, the tool being supplied in the kit; and optionally wherein the cutting tool includes a roller, the roller providing the cutting surface, or the cutting tool includes a die pad, the die pad providing the cutting surface.

72. The kit according to claim 67, wherein the bearing surface comprises an alignment feature for positioning the tissue graft before use of the cutting surface to prepare the tissue graft.

73. The kit according to claim 67, wherein the bearing surface is a substantially convex surface and optionally, wherein the cutting surface is a substantially flat surface; or
wherein the bearing surface is a substantially flat surface, and optionally, wherein the cutting surface is a substantially convex surface.
